(19) United States
(12) Patent Application Publication
(10) Pub. No.: US 2026/0175841 A1
(43) Pub. Date: Jun. 25, 2026

(54) COMPUTING A MULTI-DIMENSIONAL PERSONALIZED ADAPTIVE CRUISE CONTROL PARAMETER

(71) Applicant: TOYOTA MOTOR ENGINEERING & MANUFACTURING NORTH AMERICA, INC., Plano, TX (US)

(72) Inventors: Rohit Gupta, Santa Clara, CA (US); Ahmadreza Moradipari, Mountain View, CA (US); Amr Abdelraouf, Redwood City, CA (US); Kyungtae Han, Palo Alto, CA (US)

(73) Assignees: TOYOTA MOTOR ENGINEERING & MANUFACTURING NORTH AMERICA, INC., Plano, TX (US); TOYOTA JIDOSHA KABUSHIKI KAISHA, TOYOTA-SHI (JP)

(21) Appl. No.: 18/988,587

(22) Filed: Dec. 19, 2024

(51) Int. Cl. B60W 30/16; A61B 5/00; B60W 50/00; B60W 50/08

(52) U.S. Cl. CPC B60W 30/16; A61B 5/6893; B60W 50/0098; B60W 50/08; A61B 2503/22; B60W 2050/0012; B60W 2420/403; B60W 2520/10; B60W 2540/22; B60W 2540/221; B60W 2552/05; B60W 2554/802; B60W 2555/20; B60W 2720/10; B60W 2754/30

(57) ABSTRACT

Systems and methods are provided for improved/personalized operation of Adaptive Cruise Control (ACC). An example method may comprise: (1) computing an ACC parameter personalized for a driver of a vehicle, wherein the personalized ACC parameter comprises a multi-dimensional vector reflecting preferred following gaps for the driver across different human contexts (e.g., physiological and psychological states of the driver) and environmental contexts (e.g., weather conditions, times of day, types of traffic scenario, types of road); (2) during at least a portion of ACC operation of the vehicle, using the personalized ACC parameter to compute a target following gap for following a second vehicle; and (3) controlling speed of the vehicle in accordance with the target following gap.

### TECHNICAL FIELD

The present disclosure relates generally to the field of advanced driver-assistance systems (ADAS), and more particularly some implementations relate to systems and methods for computing a multi-dimensional personalized adaptive cruise control (ACC) parameter for a driver.

### BACKGROUND OF THE INVENTION

A cruise control system can automatically control speed of a vehicle. For example, a driver of the vehicle may use the cruise control system to set a desired speed and the cruise control system can control the throttle of the vehicle to maintain the speed without any outside intervention (e.g., the driver pressing the accelerator pedal). In various implementations, settings of the cruise control system may be controlled by the driver using various buttons and may be deactivated by pressing a brake pedal in the vehicle.

An evolution of the cruise control system is the adaptive cruise control (ACC) system. ACC may refer to a type of advanced driver-assistance system (ADAS) that utilizes sensors of a vehicle to adapt cruise control to changing conditions around the vehicle.

For example, an ACC system may control a vehicle to maintain a user-defined/set speed when a road ahead is clear, and switch to a following gap-based control (e.g., a time gap-based control, a distance gap-based control, or a combination thereof) when vehicle sensors (e.g., cameras, proximity sensors such as Radar, Lidar, sonar, etc.) detect another vehicle or obstacle ahead of the vehicle. For example, the ACC system may maintain a pre-determined following gap (e.g., 3 seconds, 100 feet, etc.) behind another vehicle instead of maintaining a pre-determined speed.

### BRIEF SUMMARY OF THE DISCLOSURE

According to various aspects of the disclosed technology, systems and methods for improved/personalized ACC operation are provided.

An example method may comprise: (1) computing an Adaptive Cruise Control (ACC) parameter personalized for a driver of a vehicle, wherein the personalized ACC parameter comprises a multi-dimensional vector reflecting preferred following gaps for the driver across different human contexts and environmental contexts; (2) during at least a portion of ACC operation of the vehicle, using the personalized ACC parameter to compute a target following gap for following a second vehicle; and (3) controlling speed of the vehicle in accordance with the target following gap.

In certain embodiments of the method, the multi-dimensional vector may comprise: (a) one or more dimensions reflecting the human contexts; and (b) one or more dimensions reflecting the environmental contexts. In some embodiments, the multi-dimensional vector may have different values at different vehicle speeds.

In various embodiments of the method, the one or more dimensions reflecting the human contexts may comprise one or more dimensions reflecting at least one of: (i) physiological states of the driver; and (ii) psychological states of the driver. In some of such embodiments, computing the personalized ACC parameter for the driver may comprise computing the personalized ACC parameter dynamically based on: (A) real-time speeds of the vehicle and corresponding following gaps behind other vehicles when the ACC is enabled; (B) real-time driving inputs provided by the driver; (C) real-time environmental conditions; and (D) at least one of real-time physiological states of the driver and real-time psychological states of the driver. Relatedly, using the personalized ACC parameter to compute the target following gap for following the second vehicle may comprise using a feed forward function to compute the target following gap based on: (A) the personalized ACC parameter; (B) a current speed of the vehicle; (C) current environmental conditions; and (D) at least one of a current physiological state of the driver and a current psychological state of the driver.

In some embodiments of the method, the one or more dimensions reflecting the environmental contexts may comprise one or more dimensions reflecting at least one of: (i) times of day; (ii) weather conditions; (iii) traffic scenarios; and (iv) road types.

In various embodiments of the method, the personalized ACC parameter may comprise one or more safety-related boundaries.

A first example vehicle of the presently disclosed technology may comprise: (1) one or more processors; and (2) memory storing instructions, which when executed by the one or more processors, cause the vehicle to perform operations, the operations comprising: (a) computing an Adaptive Cruise Control (ACC) parameter personalized for a driver of the vehicle, wherein the personalized ACC parameter comprises a multi-dimensional vector reflecting preferred following gaps for the driver across different human contexts and environmental contexts; (b) during at least a portion of ACC operation of the vehicle, using the personalized ACC parameter to compute a target following gap for following a second vehicle; and (c) controlling speed of the vehicle in accordance with the target following gap.

In some embodiments of the first vehicle, the multi-dimensional vector may comprise: (i) one or more dimensions reflecting the human contexts; and (ii) one or more dimensions reflecting the environmental contexts.

In certain embodiments of the first vehicle, the one or more dimensions reflecting the human contexts may comprise one or more dimensions reflecting at least one of: (A) physiological states of the driver; and (B) psychological states of the driver. In some of such embodiments, the operations may further comprise: (1) receiving, from a device of the driver, biometric data of the driver; and (2) based on the received biometric data, determining at least one of the physiological states of the driver and the psychological states of the driver. In other embodiments, the vehicle may further comprise one or more in-cabin sensors and the operations may further comprise: (1) receiving, from the one or more in-cabin sensors, biometric data of the driver; and (2) based on the received biometric data, determining at least one of the physiological states of the driver and the psychological states of the driver. The one or more in-cabin sensors may comprise at least one of: (a) a camera positioned to capture facial expressions of the driver; (b) an electromyography sensor; and (c) a heart rate sensor.

In various embodiments of the first vehicle, the one or more dimensions reflecting the environmental contexts may comprise one or more dimensions reflecting at least one of: (A) times of day; (B) weather conditions; (C) traffic scenarios; and (D) road types.

In some embodiments of the first vehicle, the multi-dimensional vector may comprises different values at different vehicle speeds and computing the personalized ACC parameter for the driver comprises computing the personalized ACC parameter dynamically based on: (i) real-time speeds of the vehicle and corresponding following gaps behind other vehicles when the ACC is enabled; (ii) real-time driving inputs provided by the driver; (iii) real-time environmental conditions; and (iv) at least one of real-time physiological states of the driver and real-time psychological states of the driver. Relatedly, using the personalized ACC parameter to compute the target following gap for following the second vehicle may comprise using a feed forward function to compute the target following gap based on: (i) the personalized ACC parameter; (ii) a current speed of the vehicle; (iii) current environmental conditions; and (iv) at least one of a current physiological state of the driver and a current psychological state of the driver.

A second example vehicle may comprise: (1) one or more in-cabin sensors; (2) one or more processors; and (3) memory storing instructions, which when executed by the one or more processors, cause the vehicle to perform operations, the operations comprising: (i) during operation of adaptive cruise control (ACC) in the vehicle, dynamically receiving: (a) real-time biometric data of a driver of the vehicle from the one or more in-cabin sensors, (b) real-time environmental condition data, (c) real-time data indicating speeds of the vehicle and following gaps following other vehicles, and (d) real-time driving inputs provided by the driver; (ii) correlating (a)-(d) to generate time series data reflecting preferred following gaps for the driver at different vehicle speeds across different human contexts and environmental contexts; and (iii) based on the time series data, dynamically computing an ACC parameter personalized for the driver, wherein the personalized ACC parameter comprises a multi-dimensional vector reflecting the preferred following gaps for the driver at different vehicle speeds across different human contexts and environmental contexts.

In some embodiments of the second vehicle, the operations may further comprise dynamically determining, based on the received biometric data, at least one of: (A) real-time physiological states of the driver; and (B) real-time psychological states of the driver.

In various embodiments of the second vehicle, the one or more in-cabin sensors may comprise at least one of: (1) a camera positioned to capture facial expressions of the driver; (2) an electromyography sensor; and (3) a heart rate sensor.

Other features and aspects of the disclosed technology will become apparent from the following detailed description, taken in conjunction with the accompanying drawings, which illustrate, by way of example, the features in accordance with applications of the disclosed technology. The summary is not intended to limit the scope of any inventions described herein, which are defined solely by the claims attached hereto.

### BRIEF DESCRIPTION OF THE DRAWINGS

The present disclosure, in accordance with one or more various applications, is described in detail with reference to the following figures. The figures are provided for purposes of illustration only and merely depict examples.

FIG. 1 depicts an example computing system with which embodiments of the presently disclosed technology may be implemented.

FIG. 2 depicts an example vehicle with which embodiments of the presently disclosed technology may be implemented.

FIG. 3 depicts an example system for computing a multi-dimensional personalized ACC parameter and using the multi-dimensional personalized ACC parameter to compute a target following gap, in accordance with various embodiments of the presently disclosed technology.

FIG. 4 depicts an example graph illustrating a process for computing a multi-dimensional personalized ACC parameter, in accordance with various embodiments of the presently disclosed technology.

FIG. 5 depicts an example process for computing a multi-dimensional personalized ACC parameter and using the multi-dimensional personalized ACC parameter to compute a target following gap, in accordance with various embodiments of the presently disclosed technology.

FIG. 6 depicts an example process for computing a multi-dimensional personalized ACC parameter, in accordance with various embodiments of the presently disclosed technology.

FIG. 7 depicts an example process for computing a multi-dimensional personalized ACC parameter and using the multi-dimensional personalized ACC parameter to compute a target following gap, in accordance with various embodiments of the presently disclosed technology.

FIG. 8 is an example illustration of a computing component that may be used to implement various features of embodiments described in the present disclosure.

The figures are not exhaustive and do not limit the present disclosure to the precise form disclosed.

### DETAILED DESCRIPTION

As described above, ACC may refer to a type of ADAS that utilizes sensors of a vehicle to adapt cruise control to changing conditions around the vehicle.

For example, an ACC system may control a vehicle to maintain a user-defined/set speed when a road ahead is clear, and switch to a following gap-based control when vehicle sensors detect another vehicle or obstacle ahead of the vehicle.

Despite the potential benefits of ACC, issues still exist with its acceptance by drivers. This is in part because many ACC systems fail to adequately adapt to the unique preferences/styles of different drivers.

While some nascent ACC systems attempt to personalize ACC operation, such systems tend to be computationally/resource intensive—which can increase implementation costs. For example, certain ACC systems leverage reinforcement learning (RL) and cost function minimization to: (1) learn a driver's cruise control preferences/tendencies; and (2) predict/interpolate cruise control outputs based on the learned preferences/tendencies. These RL-based systems can require significant time, data, and computation/memory resources for machine learning (ML) model training. Relatedly, even after ML model training, dynamically computing/minimizing a cost function to determine cruise control outputs can also be very computationally intensive.

Other attempts at personalizing ACC have different limitations. For example, certain ACC systems may not consider fully consider human contexts (e.g., a driver's physiological or psychological state) and environment contexts (e.g., weather conditions, time of day, type of traffic scenario, road type, etc.) when personalizing ACC operation to a driver. For instance, a respective driver's preferred following gaps may vary based on their mood, level of alertness, time of day (e.g., before or after work), weather conditions (e.g., sunny vs. rainy/snowy), etc. Accordingly, drivers may not perceive these ACC systems as realizing/matching their desired following gaps across all human and environmental contexts—which can lead to distrust (and reduced use) of such ACC systems.

Against this backdrop, the presently disclosed technology provides computationally efficient systems and methods that personalize ACC operation across a wide spectrum of human and environmental contexts.

For example, a vehicle of the presently disclosed technology may be configured to: (1) dynamically compute a multi-dimensional vector reflecting preferred following gaps for a driver across different human contexts and environmental contexts (i.e., a multi-dimensional personalized ACC parameter); (2) use the multi-dimensional personalized ACC parameter to compute a target following gap for following a second vehicle (e.g., by utilizing a feed forward function that incorporates the multi-dimensional personalized ACC parameter and takes current/detected human and environmental contexts as inputs); and (3) control speed of the vehicle in accordance with the target following gap.

Leveraging a simple/elegant parametric approach to compute and then utilize the above-described multi-dimensional personalized ACC parameter, the vehicle can personalize ACC operation across human and environmental contexts more rapidly, and with fewer computation/memory resources than alternative technologies that rely on, e.g., complex RL training, dynamically minimizing a cost function, predicting/interpolating cruise control outputs based on the foregoing, etc.

Moreover, the vehicle may realize/match a driver's desired following gaps more consistently than alternative technologies which do not fully consider human and environmental contexts when personalizing ACC operation.

For example, the above-described multi-dimensional personalized ACC parameter may comprise: (a) one or more dimensions reflecting human contexts; and (b) one or more dimensions reflecting environmental contexts.

The dimension(s) reflecting human contexts may reflect at least one of: (i) physiological states of the driver; and (ii) psychological states of the driver. In some implementations, the vehicle may comprise in-cabin sensors that obtain biometric data of the driver indicative of the driver's physiological/psychological states (e.g., cameras positioned to capture facial expressions of the driver, heart rate sensors, electromyography sensors, etc.). In other implementations, the vehicle may receive biometric data of the driver from a device (e.g., a wearable device) of the driver. In either case, the vehicle may automatically determine the driver's physiological/psychological states based on collected biometric data of the driver.

The dimension(s) reflecting environmental contexts may reflect at least one of: (i) times of day (e.g. morning vs. afternoon vs. night); (ii) weather conditions (e.g., sunny/clear vs. rainy/snowy); (iii) traffic scenarios (e.g., stop-and-go traffic vs. free-flowing traffic); and (iv) road types (e.g., multi-lane highway vs. single lane highway). As alluded to above, the vehicle may leverage various sensors (e.g., cameras, proximity sensors, environmental sensors, GPS sensors, etc.) and data sources (e.g., a navigation system of the vehicle, communications with other vehicles and roadside infrastructure) to detect the environmental conditions referenced above.

To effectively learn a driver's preferred following gaps across these different human and environmental contexts, the vehicle can correlate human context data and environmental context data collected within a respective time interval with: (1) contemporaneous data (i.e., data from the same time interval) indicating speeds of the vehicle and corresponding following gaps during operation of an ACC system; and (2) contemporaneous driving inputs provided by the driver during operation of the ACC system (e.g., the driver setting/pre-defining following gaps at different speeds, accelerator and brake pedal depression patterns, etc.). Such correlating may generate time-series data that the vehicle uses to dynamically compute the multi-dimensional personalized ACC parameter reflecting preferred following gaps for the driver at different vehicle speeds across a wide spectrum of human and environmental contexts. By controlling operation of the ACC system in accordance with the multi-dimensional personalized ACC parameter, the vehicle can consequently realize/match the driver's desired following gaps more consistently than alternative technologies which do not fully consider human and environmental contexts when personalizing ACC operation to the driver. Moreover (and as alluded to above), by reflecting these human and environmental contexts in a single, multi-dimensional parameter/vector used to compute a target following gap, the vehicle may be more computationally efficient than alternative technologies that rely on, e.g., complex RL training, dynamically reducing a cost function, predicting/interpolating cruise control outputs based on the foregoing, etc.

Systems and methods of the presently disclosed technology will now be described in greater detail in conjunction with the following FIGs.

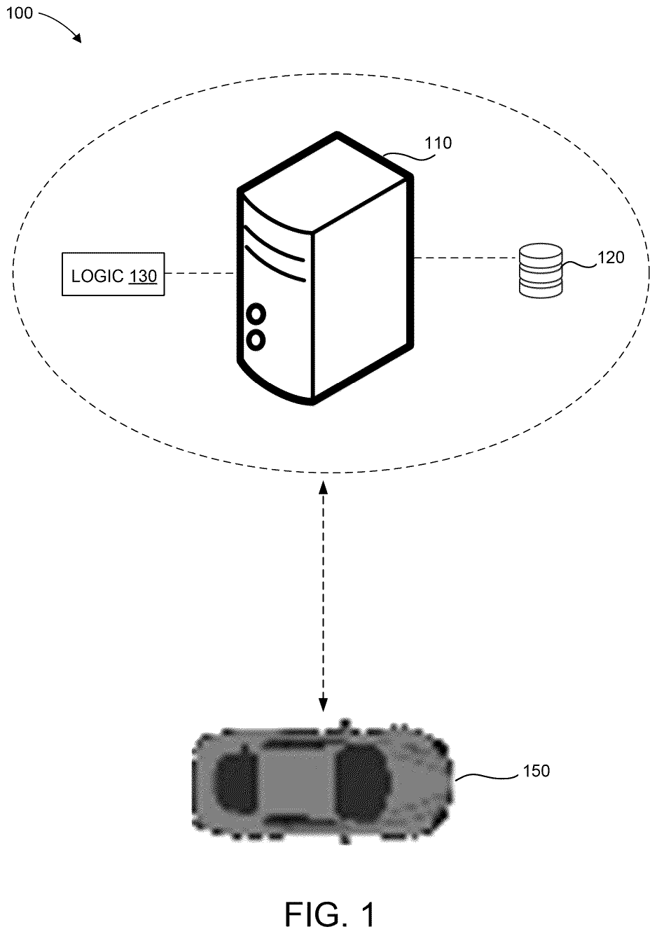

FIG. 1 depicts an example computing system 100 with which embodiments of the presently disclosed technology may be implemented. As depicted, in certain implementations computing system 100 may be implemented in whole, or in part, in a vehicle 150.

In various implementations, vehicle 150 can input data into a computing component 110. Computing component 110 may perform one or more available operations on the input data to generate outputs, such as computing a multi-dimensional personalized ACC parameter, using the multi-dimensional personalized ACC parameter to compute a target following gap, and controlling speed of vehicle 150 in accordance with the target following gap. Vehicle 150 may further display the outputs on a graphical user interface (GUI). The GUI may be in vehicle 150 or on a computing device, such as a desktop computer, a laptop, a mobile phone, a tablet device, an Internet of Things (IoT) device, etc. The GUI may display the outputs as a two-dimensional (2D) or three-dimensional (3D) layout/map showing the various outputs of computing component 110.

Computing component 110 may include one or more processors and logic 130 that implement instructions to carry out the functions of the computing component 110, for example, dynamically computing and storing a multi-dimensional personalized ACC parameter for a driver, using the multi-dimensional personalized ACC parameter to compute a target following gap, and controlling speed of vehicle 150 in accordance with the target following gap. Computing component 110 may store, in a database 120, details regarding scenarios or conditions in which some algorithms, image datasets, and assessments are performed in accordance with the foregoing.

A respective processor of computing component 110 may include one or more GPUs, CPUs, microprocessors or any other suitable processing system. The respective processor may include one or more single core or multicore processors. The respective processor may execute instructions stored in a non-transitory computer readable medium. Logic 130 may contain instructions (e.g., program logic) executable by the respective processor may to execute various functions of computing component 110. Logic 130 may contain additional instructions as well, including instructions to transmit data to, receive data from, and interact with vehicle 150.

In certain implementations, computing system 100 may implement machine learning (ML) models. As used herein, ML can refer to methods that, through the use of algorithms, are able to automatically extract intelligence or rules from training data sets and capture the same in informative models. In turn, those models can make predictions based on patterns or inferences gleaned from subsequent data input into a trained model. Certain ML algorithms disclosed herein may be supervised and/or unsupervised depending on the implementation.

Although one example computing system 100 is illustrated in FIG. 1, in various embodiments multiple computing systems 100 can be included. Additionally, one or more systems and subsystems of computing system 100 can include their own dedicated or shared computing component 110, or a variant thereof. Accordingly, although computing system 100 is illustrated as a discrete computing system, this is for ease of illustration only, and computing system 100 can be distributed among various systems or components.

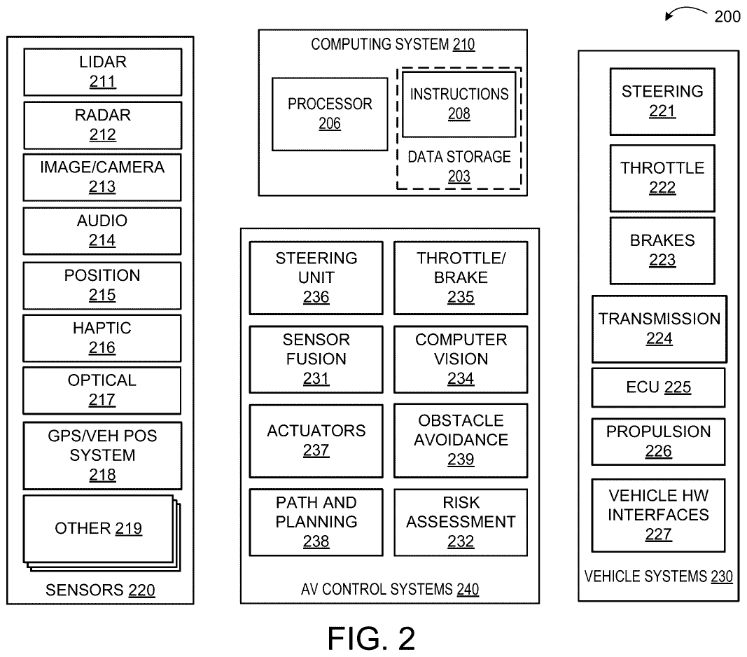

FIG. 2 depicts an example vehicle 200 with which embodiments of the presently disclosed technology may be implemented.

Vehicle 200 may comprise various types of vehicles, such as an automobile, truck, motorcycle, bicycle, scooter, moped, recreational vehicle, etc. Vehicle 200 may include autonomous, semi-autonomous, and manual operation.

Vehicle 200 may include components, such as a computing system 210, sensors 220, AV control systems 240 and vehicle systems 230. Any of computing system 210, sensors 220, AV control systems 240, and vehicle systems 230 can be part of an automated vehicle system/advanced driver assistance system (ADAS). ADAS can provide navigation control signals (i.e., control signals to actuate vehicle 200 and operate one or more vehicle systems 230 as shown in FIG. 2) for vehicle 200 to navigate a variety of situations. As used herein, ADAS can be an autonomous vehicle control system adapted for any level of vehicle control and driving autonomy. For example, the ADAS can be adapted for level 1, level 2, level 3, level 4, and level 5 autonomy (according to SAE standard). ADAS can allow for control mode blending (i.e., blending of autonomous and assisted control modes with human driver control). ADAS can correspond to a real-time machine perception system for vehicle actuation in a multi-vehicle environment. Vehicle 200 may include a greater or fewer quantity of systems and subsystems, and each could include multiple elements. Accordingly, one or more of the functions of the technology disclosed herein may be divided into additional functional or physical components, or combined into fewer functional or physical components. Additionally, although the systems and subsystems illustrated in FIG. 2 are shown as being partitioned in a particular way, the functions of vehicle 200 can be partitioned in other ways. For example, various vehicle systems and subsystems can be combined in different ways to share functionality.

Sensors 220 may include a plurality of different sensors to gather data regarding vehicle 200, its driver/operator, its operation, and its surrounding environment. Although various sensors are shown, it may be appreciated that systems and methods for detecting and responding to intervening obstacles may not require many sensors. It may also be appreciated that the systems and methods described herein can be augmented by sensors of vehicle 200. In this example, sensors 220 include a light detection and ranging (LiDAR) sensor 211, a radar sensor 212, image sensors 213 (e.g., a camera), audio sensors 214, a position sensor 215, a haptic sensor 216, an optical sensor 217, a Global Positioning System (GPS) or other vehicle positioning system 218, and other like distance measurement and environment sensing sensors 219.

As alluded to above, sensors 220 may further include sensors for collecting data related to a driver's physiological and psychological state. Such sensors may include, in-cabin image sensors, electromyography sensors, heartrate sensors, motion sensors, pressure sensors, microwave sensors, etc. The data collected by sensors 220 may be sent to a vehicle electronic control unit (ECU) or other processing unit. Sensors 220 (and other vehicle components) may be duplicated for redundancy.

Distance measuring sensors such as LiDAR sensor 211, radar sensor 212, IR sensors and other like sensors can be used to gather data to measure distances and closing rates to various external objects such as other vehicles, roads, traffic signs, pedestrians, light poles and other objects. Image sensors 213 can include one or more cameras or other image sensors to capture images of the environment around vehicle 200, such as weather and road surfaces, as well as internal to vehicle 200. Information from image sensors 213 (e.g., a camera) can be used to determine information about the environment surrounding vehicle 200 including, for example, information regarding weather, traffic conditions, time of day, road surfaces and other objects surrounding vehicle 200. For example, image sensors 213 may be able to recognize specific vehicles (e.g., color, vehicle type), landmarks or other features (including, e.g., street signs, traffic lights, etc.), slope of the road, lines on the road, damages and other potentially hazardous conditions to the road, curbs, objects to be avoided (e.g., other vehicles, pedestrians, bicyclists, etc.) and other landmarks or features. Information from image sensors 213 can be used in conjunction with other information such as map data, or information from positioning system 218 to determine, refine, or verify vehicle (ego vehicle or another vehicle) location as well as detect obstructions. Information from image sensors 213 can be used in conjunction with other information such as map data, or information from positioning system 218 to determine traffic scenarios as well.

Vehicle positioning system 218 (e.g., GPS or other positioning system) can be used to gather position information about a current location of vehicle 200 as well as other positioning or navigation information, such as the positioning information about a current location and direction of movement of vehicle 200 according to a particular road.

Other sensors 219 may be provided as well. Other sensors 219 can include vehicle acceleration sensors, vehicle speed sensors, wheelspin sensors (i.e., one for each wheel), tire pressure monitoring sensors (i.e., one for each tire), vehicle clearance sensors, left-right and front-rear slip ratio sensors, environmental sensors (i.e., to detect weather, traction conditions, or other environmental conditions), seat pressure monitoring sensors (i.e., in the driver seat to measure muscle tension of the driver), motion sensors, electromyography sensors, and microwave sensors. Other sensors 219 can be further included for a given implementation of ADAS. Various sensors 220, such as other sensors 219, may be used to provide input to computing system 210 and other systems of vehicle 200 so that the systems have information useful to compute a multi-dimensional personalized ACC parameter for a driver, use the multi-dimensional personalized ACC parameter to compute a target following gap, control speed of vehicle 200 in accordance with the target following gap, etc.

AV control systems 240 may include a plurality of different systems/subsystems to control operation of vehicle 200. In this example, AV control systems 240 can include, autonomous driving module (not shown), steering unit 236, throttle and brake control unit 235, sensor fusion module 231, computer vision module 234, path and planning module 238, obstacle avoidance module 239, risk assessment module 232 and actuator(s) 237. Sensor fusion module 231 can be included to evaluate data from a plurality of sensors, including sensors 220. Sensor fusion module 231 may use computing system 210 or its own computing system to execute algorithms to assess inputs from the various sensors.

Throttle and brake control unit 235 can be used to control actuation of throttle and braking mechanisms of vehicle 200 to accelerate, slow down, stop or otherwise adjust the speed of vehicle 200. For example, the throttle unit can control the operating speed of the engine or motor used to provide motive power for vehicle 200. Likewise, the brake unit can be used to actuate brakes (e.g., disk, drum, etc.) or engage regenerative braking (i.e., such as in a hybrid or electric vehicle) to slow or stop vehicle 200.

Steering unit 236 may include any of a number of different mechanisms to control or alter the heading of vehicle 200. For example, steering unit 236 may include the appropriate control mechanisms to adjust the orientation of the front or rear wheels of vehicle 200 to accomplish changes in direction of vehicle 200 during operation. Electronic, hydraulic, mechanical or other steering mechanisms may be controlled by steering unit 236.

Computer vision module 234 may be included to process image data (e.g., image data captured from image sensors 213, or other image data) to evaluate the environment within or surrounding vehicle 200. For example, algorithms operating as part of computer vision module 234 can evaluate still or moving images to determine features and landmarks (e.g., road pavements, lines of the road, damages and other potentially hazardous conditions on the road, road signs, traffic lights, lane markings and other road boundaries, etc.), obstacles (e.g., pedestrians, bicyclists, other vehicles, other obstructions in the path of the subject vehicle) and other objects. Computer vision module 234 can include video tracking and other algorithms to recognize objects such as the foregoing, estimate their speed, map the surroundings, and so on. Computer vision module 234 may be able to model the road traffic vehicle network, predict incoming hazards and obstacles, predict road hazard, and determine one or more contributing factors to identifying obstructions. Computer vision module 234 may be able to perform depth estimation, image/video segmentation, camera localization, and object classification according to various classification techniques (including by applied neural networks).

Path and planning module 238 may be included to compute a desired path for vehicle 200 based on input from various other sensors and systems. For example, path and planning module 238 can use information from positioning system 218, sensor fusion module 231, computer vision module 234, obstacle avoidance module 239 (described below) and other systems (e.g., AV control systems 240, sensors 220, and vehicle systems 230) to determine a safe path to navigate vehicle 200 along a segment of a desired route. Path and planning module 238 may also be configured to dynamically update vehicle 200's path as real-time information is received from sensors 220 and other control systems 240.

Obstacle avoidance module 239 can be included to determine control inputs necessary to avoid obstacles and obstructions detected by sensors 220 or AV control systems 240. Obstacle avoidance module 239 can work in conjunction with path and planning module 238 to determine an appropriate path to avoid and navigate around obstacles and obstructions.

Path and planning module 238 (either alone or in conjunction with one or more other module of AV control systems 240, such as obstacle avoidance module 239, computer vision module 234, and sensor fusion module 231) may also be configured to perform and coordinate one or more vehicle maneuvers. Example vehicle maneuvers can include at least one of a path tracking, stabilization and collision avoidance maneuver. With connected vehicles, vehicle maneuvers can be performed at least partially cooperatively between the connected vehicles to gather a sufficient amount of data of the environment, including obstructions and traffic. A sufficient amount of data of an obstruction may include collecting data of the obstruction at various angles and perspectives. Each different type of obstruction may warrant a different amount of data to be collected and analyzed to make the needed determinations to verify the obstruction and determine the condition of traffic. For example, data needed to verify a small obstruction, like a small pothole, may be minimal as the connected vehicles collecting verification data of the small pothole obstruction may only need to collect data of missing asphalt on the road. The data needed to verify a larger obstruction, like a downed traffic light, may be much more extensive as the connected vehicles collecting verification data of the downed traffic light obstruction may need to collect data of the portion of the roadway blocked by the downed traffic light, electrical issues present on the roadway, disrupted traffic flow caused by the downed traffic light, including, for example, any other vehicles or objects blocking traffic due to the downed traffic light, additional obstructions on the road caused by the downed traffic light, including, for example, cracks, potholes, debris, etc., and so on. Hence, those of ordinary skill in the art will understand what sufficient means in the context of collecting a sufficient amount of data to verify an obstruction to determine the condition of traffic.

Vehicle systems 230 may include a plurality of different systems/subsystems to control operation of vehicle 200. In this example, vehicle systems 230 include steering system 221, throttle system 222, brakes 223, transmission 224, electronic control unit (ECU) 225, propulsion system 226 and vehicle hardware interfaces 227. Vehicle systems 230 may be controlled by AV control systems 240 in autonomous, semi-autonomous or manual modes of vehicle 200. For example, in autonomous or semi-autonomous mode, AV control systems 240, alone or in conjunction with other systems, can control vehicle systems 230 to operate the vehicle in a fully or semi-autonomous fashion. When control is assumed, computing system 210 and AV control system 230 can provide vehicle control systems to vehicle hardware interfaces for controlled systems such as steering angle 221, brakes 223, throttle 222, or other hardware interfaces 227, such as traction force, turn signals, horn, lights, etc. This may also include an assist mode in which the vehicle takes over partial control or activates ADAS controls (e.g., AC control systems 240) to assist the driver with vehicle operation.

Computing system 210 in the illustrated example includes a processor 206, and memory 203. Some or all of the functions of vehicle 200 may be controlled by computing system 210. Processor 206 can include one or more GPUs, CPUs, microprocessors or any other suitable processing system. Processor 206 may include one or more single core or multicore processors. Processor 206 executes instructions 208 stored in a non-transitory computer readable medium, such as memory 203.

Memory 203 may contain instructions (e.g., program logic) executable by processor 206 to execute various functions of vehicle 200, including those of vehicle systems and subsystems. Memory 203 may contain additional instructions as well, including instructions to transmit data to, receive data from, interact with, and control one or more of the sensors 220, AV control systems 240 and vehicle systems 230. In addition to the instructions, memory 203 may store data and other information used by the vehicle and its systems and subsystems for operation, including operation of vehicle 200 in the autonomous, semi-autonomous or manual modes. For example, memory 203 can include data that has been communicated to vehicle 200 (e.g., via V2V (vehicle-to-vehicle) communication), mapping data, a model of the current or predicted road traffic vehicle network, vehicle dynamics data, computer vision recognition data, and other data which can be useful for the execution of one or more vehicle maneuvers, for example by one or more modules of the AV control systems 240.

Although one computing system 210 is illustrated in FIG. 2, in various applications multiple computing systems 210 can be included. Additionally, one or more systems and subsystems of vehicle 200 can include its own dedicated or shared computing system 210, or a variant thereof. Accordingly, although computing system 210 is illustrated as a discrete computing system, this is for ease of illustration only, and computing system 210 can be distributed among various vehicle systems or components.

Vehicle 200 may also include a (wireless or wired) communication system (not illustrated) to communicate with other vehicles, infrastructure elements, cloud components and other external entities using any of a number of communication protocols including, for example, V2V (vehicle-to-vehicle), V2I (vehicle-to-infrastructure) and V2X (vehicle-to-everything) protocols. Such a wireless communication system may allow vehicle 200 to receive information from other objects including, for example, map data, data regarding infrastructure elements, data regarding operation and intention of surrounding vehicles, and so on. A wireless communication system may allow vehicle 200 to receive updates to data that can be used to execute one or more vehicle control modes, and vehicle control algorithms as discussed herein. Wireless communication system may also allow vehicle 200 to transmit information to other objects and receive information from other objects (such as other vehicles, user devices, or infrastructure). In some applications, one or more communication protocol or dictionaries can be used, such as the SAE J2935 V2X Communications Message Set Dictionary. In some applications, the communication system may be useful in retrieving and sending one or more data useful in detecting and verifying obstructions, as disclosed herein.

Communication system can be configured to receive data and other information from sensors 220 that is used in determining whether and to what extent control mode blending should be activated. Additionally, communication system can be used to send an activation signal or other activation information to various vehicle systems 230 and AV control systems 240 as part of controlling vehicle 200. For example, communication system can be used to send signals to one or more of the vehicle actuators 237 to control parameters, for example, maximum steering angle, throttle response, vehicle braking, torque vectoring, and so on.

In some applications, computing functions for various applications disclosed herein may be performed entirely on computing system 210, distributed among two or more computing systems 210 of vehicle 200, performed on a cloud-based platform, performed on an edge-based platform, or performed on a combination of the foregoing.

Path and planning module 238 can allow for executing one or more vehicle control mode(s), and vehicle control algorithms in accordance with various implementations of the systems and methods disclosed herein.

In operation, path and planning module 238 (e.g., by a driver intent estimation module, not shown) can receive information regarding human control input used to operate vehicle 200. As described above, information from sensors 220, actuators 237 and other systems can be used to determine the type and level of human control input. Path and planning module 238 can use this information to predict driver action. Path and planning module 238 can use this information to generate a predicted path and model the road traffic vehicle network. This may be useful in evaluating road conditions, determining and verifying obstructions, and determining traffic conditions. As also described above, information from sensors, and other systems can be used to evaluate road conditions, determine and verify obstructions, and determine traffic conditions. Eye state tracking, attention tracking, or intoxication level tracking, for example, can be used to determine physiological and psychological state of a driver. It may be appreciated the driver's physiological and psychological state can contribute to computing a multi-dimensional personalized ACC parameter as disclosed herein.

Path and planning module 238 can receive state information such as, for example from visibility maps, traffic and weather information, hazard maps, and local map views. Information from a navigation system can also provide a mission plan including maps and routing to path and planning module 238.

The path and planning module 238 (e.g., by a driver intent estimation module, not shown) can receive this information and predict behavior characteristics within a future time horizon. This information can be used by path and planning module 238 for executing one or more planning decisions. Planning decisions can be based on one or more policy (such as defensive driving policy). Planning decisions can be based on one or more level of autonomy, connected vehicle actions, one or more policy (such as defensive driving policy, cooperative driving policy, such as swarm or platoon formation, leader following, etc.). Path and planning module 238 can generate an expected model for the road traffic hazards and assist in creating a predicted traffic hazard level and verification strategy for vehicles to implement.

Path and planning module 238 can receive risk information from risk assessment module 232. Path and planning module 238 can receive vehicle capability and capacity information from one or more vehicle systems 230. Vehicle capability can be assessed, for example, by receiving information from vehicle hardware interfaces 229 to determine vehicle capabilities and identify a reachable set model. Path and planning module 238 can receive surrounding environment information (e.g., from computer vision module 234, and obstacle avoidance module 239). Path and planning module 238 can apply risk information and vehicle capability and capacity information to trajectory information (e.g., based on a planned trajectory and driver intent) to determine a safe or optimized trajectory for vehicle 200 given the driver's intent, policies (e.g., safety or vehicle cooperation policies), communicated information, given one or more obstacles in the surrounding environment, road conditions, traffic conditions, etc. This trajectory information can be provided to controller (e.g., ECU 225) to provide partial or full vehicle control in the event of a risk level above threshold. A signal from risk assessment module 232 can be used generate countermeasures described herein. A signal from risk assessment module 232 can trigger ECU 225 or another AV control system 230 to take over partial or full control of the vehicle.

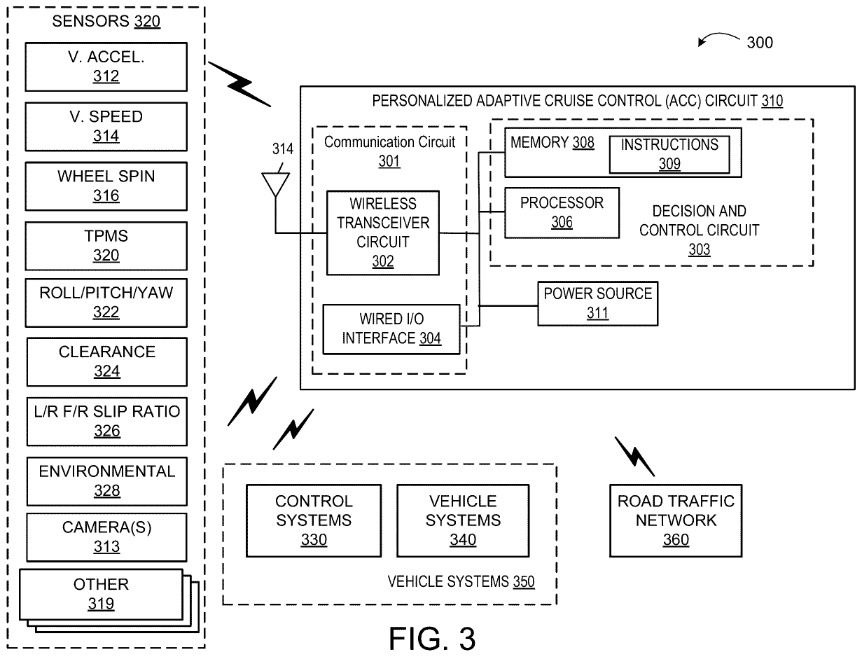

FIG. 3 depicts an example personalized ACC system 300 for computing a multi-dimensional personalized ACC parameter, in accordance with various embodiments of the presently disclosed technology.

As depicted, personalized ACC system 300 includes a personalized ACC circuit 310, a plurality of sensors 320, and a plurality of vehicle systems 350. Also included are various elements of a road traffic network 360 with which the personalized ACC system 300 can communicate. It may be appreciated that road traffic network 360 can include various elements that are navigating and important in navigating a road traffic network, such as vehicles, pedestrians (with or without connected devices that can include aspects of personalized ACC system 300 disclosed herein), or infrastructure (e.g., traffic signals, sensors, such as traffic cameras, databases, central servers, weather sensors). Other elements of road traffic network 360 can include connected elements at workplaces, or the home (such as vehicle chargers, connected devices, appliances, etc.).

Personalized ACC system 300 can be implemented as and include one or more components of the vehicle 200 shown in FIG. 2. Sensors 320, vehicle systems 350, and elements of road traffic network 360, can communicate with personalized ACC circuit 310 via a wired or wireless communication interface. As previously alluded to, elements of road traffic network 360 can correspond to connected or unconnected devices, infrastructure (e.g., traffic signals, sensors, such as traffic cameras, weather sensors), vehicles, pedestrians, obstacles, etc. that are in a broad or immediate vicinity of an ego-vehicle (e.g., vehicle 150, vehicle 200) or otherwise important to the navigation of the road traffic network (such as remote infrastructure). Although sensors 320, vehicle systems 350, and road traffic network 360, are depicted as communicating with personalized ACC circuit 310, they can also communicate with each other, as well as with other vehicle systems 350 and directly with element of a road traffic network 360. Data as disclosed herein can be communicated to and from the personalized ACC circuit 310. For example, various infrastructure (example element of road traffic network 360) can include one or more databases, such as vehicle crash data or weather data. This data can be communicated to the circuit 310, and such data can be updated based on outcomes for one or more maneuvers or navigation of the road traffic network, vehicle telematics, driver state (physiological and psychological), vehicle data from sensors 320 (e.g., tire pressure or brake status) from the vehicle. Similarly, traffic data, vehicle state data, time of travel, demographics data for drivers can be retrieved and updated. All of this data can be included in and contribute to predictive analytics (e.g., by machine learning) of accident possibility, and determinations of road conditions and poor, hazard road conditions. Similarly, models, circuits, and predictive analytics can be updated according to various outcomes.

Personalized ACC circuit 310 can evaluate road conditions, detect an obstruction, determine traffic conditions, determine driver physiological and psychological state, and compute a multi-dimensional personalized ACC parameter as described herein. As will be described in more detail herein, the detection of obstructions, evaluation of road conditions, determination of traffic conditions and determination of driver physiological and psychological state can have one or more contributing factors. Various sensors 320, vehicle systems 350, and road traffic network 360 elements may contribute to gathering data for evaluating road conditions, detecting obstructions, determining traffic conditions, and determining driver physiological and psychological state. For example, personalized ACC circuit 310 can include at least one of an obstruction detection and response circuit. Personalized ACC circuit 310 can be implemented as an ECU or as part of an ECU such as, for example electronic control unit 225. In other applications, personalized ACC circuit 310 can be implemented independently of the ECU, for example, as another vehicle system.

Personalized ACC circuit 310 can be configured to evaluate road conditions, detect obstructions, determine traffic conditions, determine driver physiological and psychological state, and dynamically compute a multi-dimensional personalized ACC parameter. Personalized ACC circuit 310 may include a communication circuit 301 (including either or both of a wireless transceiver circuit 302 with an associated antenna 314 and wired input/output (I/O) interface 304 in this example), a decision and control circuit 303 (including a processor 306 and memory 308 in this example) and a power source 311 (which can include power supply). It may be appreciated that the disclosed personalized ACC circuit 310 can be compatible with and support one or more standard or non-standard messaging protocols.

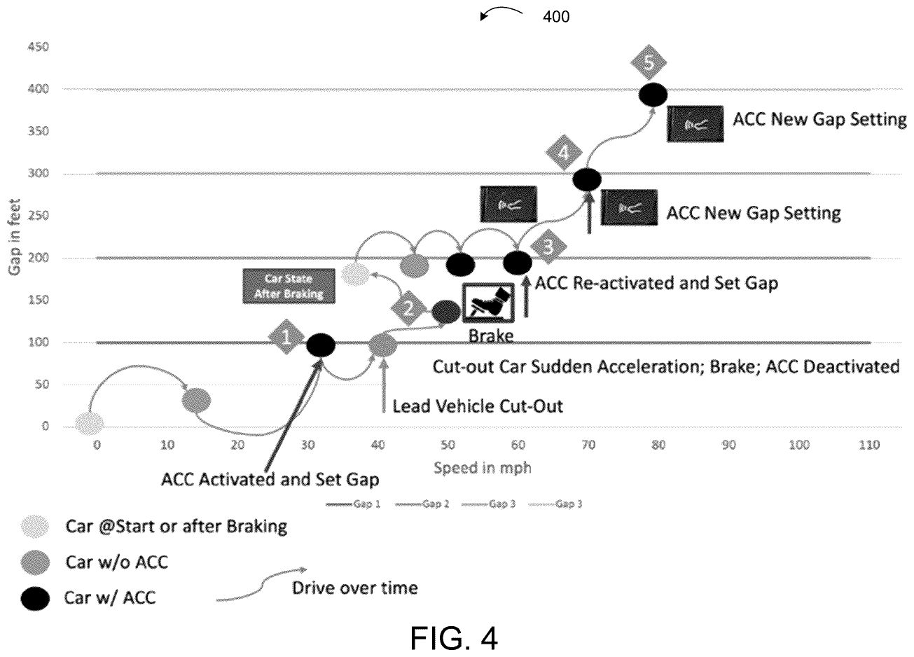

Components of personalized ACC circuit 310 are illustrated as communicating with each other via a data bus, although other communication in interfaces can be included. Decision and control circuit 303 can be configured to control one or more aspects of obstruction detection and response. Decision and control circuit 303 can be configured to execute one or more steps described with reference to FIGS. 4-6.

Processor 306 can include a GPU, CPU, microprocessor, or any other suitable processing system. Memory 308 may include one or more various forms of memory or data storage (e.g., flash, RAM, etc.) that may be used to store the calibration parameters, images (analysis or historic), point parameters, instructions and variables for processor 306 as well as any other suitable information. Memory 308 can be made up of one or more modules of one or more different types of memory, and may be configured to store data and other information as well as operational instructions 309 that may be used by the processor 306 to execute one or more functions of personalized ACC circuit 310. For example, data and other information can include vehicle driving data, such as a determined familiarity of the driver with driving and the vehicle. The data can also include values for signals of one or more sensors 320 useful in evaluating road conditions, detecting obstructions, determining traffic conditions, determining driver physiological and psychological state, and computing a multi-dimensional personalized ACC parameter. Operational instruction 309 can contain instructions for executing logical circuits, models, and methods as described herein.

Although the Example of FIG. 3 is illustrated using processor and memory circuitry, as described below with reference to circuits disclosed herein, decision and control circuit 303 can be implemented utilizing any form of circuitry including, for example, hardware, software, or a combination thereof. By way of further example, one or more processors, controllers, ASICs, PLAs, PALs, CPLDs, FPGAs, logical components, software routines or other mechanisms might be implemented to make up personalized ACC circuit 310. Components of decision and control circuit 303 can be distributed among two or more decision and control circuits 303, performed on other circuits described with respect to personalized ACC circuit 310, be performed on devices (such as cell phones) performed on a cloud-based platform (e.g., part of infrastructure), performed on distributed elements of road traffic network 360, such as at multiple vehicles, user device, central servers, performed on an edge-based platform, and performed on a combination of the foregoing.

Communication circuit 301 may include either or both a wireless transceiver circuit 302 with an associated antenna 314 and a wired I/O interface 304 with an associated hardwired data port (not illustrated). As this example illustrates, communications with personalized ACC circuit 310 can include either or both wired and wireless communications circuits 301. Wireless transceiver circuit 302 can include a transmitter and a receiver (not shown), e.g., an obstruction detection and verification broadcast mechanism, to allow wireless communications via any of a number of communication protocols such as, for example, WiFi (e.g., IEEE 802.11 standard), Bluetooth, near field communications (NFC), Zigbee, and any of a number of other wireless communication protocols whether standardized, proprietary, open, point-to-point, networked or otherwise. Antenna 314 is coupled to wireless transceiver circuit 302 and is used by wireless transceiver circuit 302 to transmit radio signals wirelessly to wireless equipment with which it is connected and to receive radio signals as well. These RF signals can include information of almost any sort that is sent or received by road condition detection and verification circuit 310 to/from other components of the vehicle, such as sensors 320, vehicle systems 350, infrastructure (e.g., servers cloud-based systems), and other devices or elements of road traffic network 360. These RF signals can include information of almost any sort that is sent or received by personalized ACC circuit 310

Wired I/O interface 304 can include a transmitter and a receiver (not shown) for hardwired communications with other devices. For example, wired I/O interface 304 can provide a hardwired interface to other components, including sensors 320 and vehicle systems 350. Wired I/O interface 304 can communicate with other devices using Ethernet or any of a number of other wired communication protocols whether standardized, proprietary, open, point-to-point, networked or otherwise.

Power source 311 such as one or more of a battery or batteries (such as, e.g., Li-ion, Li-Polymer, NiMH, NiCd, NiZn, and NiH2, to name a few, whether rechargeable or primary batteries), a power connector (e.g., to connect to vehicle supplied power, another vehicle battery, alternator, etc.), an energy harvester (e.g., solar cells, piezoelectric system, etc.), or it can include any other suitable power supply. It may be appreciated that power source 311 can be coupled to a power source of a vehicle, such as a battery and alternator. Power source 311 can be used to power personalized ACC circuit 310.

Sensors 320 can include one or more of the previously mentioned sensors 220 of FIG. 2. Sensors 320 can include one or more sensors that may or not otherwise be included on a standard vehicle (e.g., vehicle 200) with which personalized ACC circuit 310 is implemented. In the illustrated example, sensors 320 include vehicle acceleration sensors 312, vehicle speed sensors 314, wheelspin sensors 316 (e.g., one for each wheel), a tire pressure monitoring system (TPMS) 320, accelerometers such as a 3-axis accelerometer 322 to detect roll, pitch and yaw of the vehicle, vehicle clearance sensors 324, left-right and front-rear slip ratio sensors 326, environmental sensors 328 (e.g., to detect weather, salinity or other environmental conditions), and camera(s) 313 (e.g., front rear, side, top, bottom facing). Additional sensors 319 can also be included as may be appropriate for a given implementation of personalized ACC system 300.

Vehicle systems 350 can include any of a number of different vehicle components or subsystems used to control or monitor various aspects of the vehicle and its performance. For example, it can include any or all of the aforementioned vehicle systems 230 and control systems 240 shown in FIG. 2. In this example, the vehicle systems 350 may include a GPS or other vehicle positioning system 218.

During operation, personalized ACC circuit 310 can receive information from various vehicle sensors 320, vehicle systems 350, and road traffic network 360 to evaluate road conditions, detect obstructions, determine traffic conditions, determine driver physiological and psychological state, and compute a multi-dimensional personalized ACC parameter. Also, the driver/operator of the vehicle may manually trigger one or more processes described herein for evaluating road conditions, detecting obstructions, determining traffic conditions, determining driver physiological and psychological state, and computing a multi-dimensional personalized ACC parameter. Communication circuit 301 can be used to transmit and receive information between personalized ACC circuit 310, sensors 320 and vehicle systems 350. Also, sensors 320 and personalized ACC circuit 310 may communicate with vehicle systems 350 directly or indirectly (e.g., via communication circuit 301 or otherwise). Communication circuit 301 can be used to transmit and receive information between personalized ACC circuit 310, one or more other systems of a vehicle (e.g., vehicle 200), but also other elements of road traffic network 360, such as vehicles, devices (e.g., mobile phones), systems, networks (such as a communications network and central server), and infrastructure.

In various applications, communication circuit 301 can be configured to receive data and other information from sensors 320 and vehicle systems 350 that is used evaluating road conditions, detecting obstructions, determining traffic conditions, determining driver physiological and psychological state, and computing a multi-dimensional personalized ACC parameter. As one example, when data is received from an element of road traffic network 360 (such as from a driver's user device), communication circuit 301 can be used to send an activation signal and activation information to one or more vehicle systems 350 or sensors 320 for the vehicle to determine traffic conditions. For example, it may be useful for vehicle systems 350 or sensors 320 to provide data useful in determining traffic conditions. Alternatively, personalized ACC circuit 310 can be continuously receiving information from vehicle system 350, sensors 320, other vehicles, devices and infrastructure (e.g., those that are elements of road traffic network 360). Further, upon detecting an obstruction, communication circuit 301 can send a signal to other components of the vehicle, infrastructure, or other elements of the road traffic network based on the detection of the obstruction. For example, the communication circuit 301 can send a signal to a vehicle system 350 that indicates a control input for performing one or more vehicle movement patterns to navigate around the obstruction according to the type of road condition. In some applications upon detecting an obstruction, depending on the type of road condition, the driver's control of the vehicle can be prohibited, and control of the vehicle can be offloaded to the ADAS. In more specific examples, upon detection of an obstruction (e.g., by sensors 320, and vehicle system 350 or by elements of the road traffic network 360), one or more signals can be sent to a vehicle system 350, so that an assist mode can be activated and the vehicle can control one or more of vehicle systems 230 (e.g., steering system 221, throttle system 222, brakes 223, transmission 224, ECU 225, propulsion system 226, suspension, and powertrain).

The examples of FIGS. 2 and 3 are provided for illustration purposes only as examples of vehicle 200 and personalized ACC system 300 with which applications of the disclosed technology may be implemented. One of ordinary skill in the art reading this description will understand how the disclosed applications can be implemented with other vehicle platforms.

FIG. 4 depicts an example graph 400 illustrating a process for computing a multi-dimensional personalized ACC parameter, in accordance with various embodiments of the presently disclosed technology.

As depicted, graph 400 plots a distance gap (in feet) as a function of speed (in miles per hour (mph)) for an example vehicle.

Also depicted in graph 400 are five driving inputs provided by a driver of the vehicle. For example, driving input <1> may involve the driver activating an ACC system in the vehicle and setting a distance gap of 100 ft when the vehicle is traveling at approximately 30 mph. Driving input <2> may involve the driver depressing a brake pedal in the vehicle to deactivate the ACC system when the distance gap is at 100 ft and speed of the vehicle reaches approximately 40 mph. Driving input <3> may involve the driver re-activating the ACC system in the vehicle and setting a distance gap of 200 ft when the vehicle is traveling at approximately 60 mph. Driving input <4> may involve the driver resetting the distance gap to 300 ft when the vehicle reaches approximately 70 mph. Driving input <5> may involve the driver resetting the distance gap to 400 ft when the vehicle reaches approximately 80 mph.

As alluded to above, a vehicle/system of the presently disclosed technology may monitor these driving inputs in real-time along with contemporaneous human contexts (e.g., the driver's physiological and psychological states) and environmental contexts (e.g., time of day, weather conditions, traffic scenario, type of road, etc.) when the driver is providing driving inputs <1>-<5>. Based on such monitored data, the vehicle/system may dynamically compute a multi-dimensional personalized ACC parameter for the driver that reflects the driver's preferred following gaps at different speeds and across a wide spectrum of human and environmental contexts.

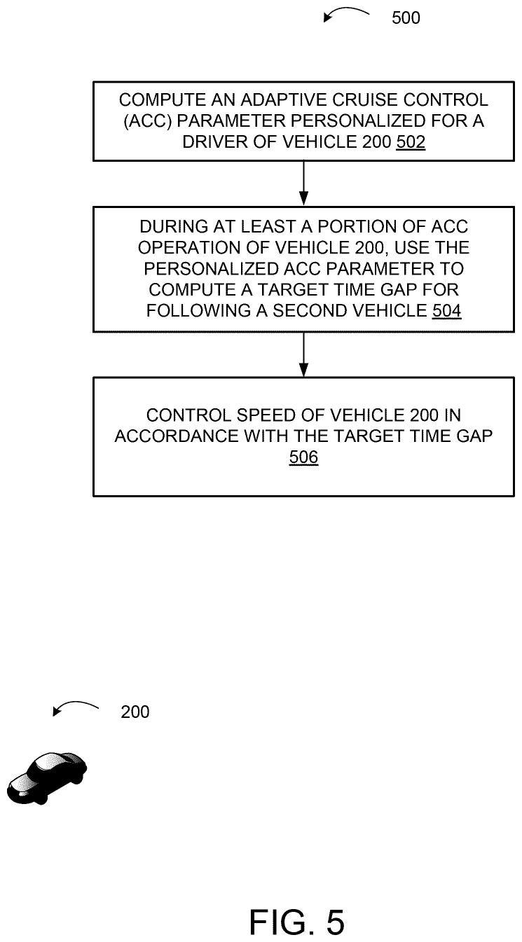

FIG. 5 depicts an example process 500 for computing a multi-dimensional personalized ACC parameter and using the multi-dimensional personalized ACC parameter to compute a target following gap, in accordance with various embodiments of the presently disclosed technology. As depicted, vehicle 200 from FIG. 2 may be configured to perform process 500.

For example, vehicle 200 can perform operation 502 to compute an ACC parameter personalized for a driver of vehicle 200 (i.e., a personalized ACC parameter). As described above, the personalized ACC parameter may comprise a multi-dimensional vector reflecting preferred following gaps for the driver at across different human contexts and environmental contexts. In various implementations, the multi-dimensional vector may comprise different values at different speeds of vehicle 200 as well.

In some implementations, the personalized ACC parameter/multi-dimensional vector may comprise: (a) one or more dimensions reflecting human contexts; and (b) one or more dimensions reflecting environmental contexts.

The dimension(s) reflecting human contexts may reflect at least one of: (i) physiological states of the driver; and (ii) psychological states of the driver. In some implementations, vehicle 200 may comprise in-cabin sensors that obtain biometric data of the driver indicative of the driver's physiological/psychological states (e.g., cameras positioned to capture facial expressions of the driver, heart rate sensors, electromyography sensors, etc.). In other implementations, vehicle 200 may receive biometric data of the driver from a device (e.g., a wearable device) of the driver. In either case, vehicle 200 may automatically determine the driver's physiological/psychological states based on collected biometric data of the driver.

The dimension(s) reflecting environmental contexts may reflect at least one of: (i) times of day (e.g. morning vs. afternoon vs. night); (ii) weather conditions (e.g., sunny/clear vs. rainy/snowy); (iii) traffic scenarios (e.g., stop-and-go traffic vs. free-flowing traffic); and (iv) road types (e.g., multi-lane highway vs. single lane highway). As alluded to above, vehicle 200 may leverage various sensors (e.g., cameras, proximity sensors, environmental sensors, GPS sensors, etc.) and data sources (e.g., a navigation system of vehicle 200, communications with other vehicles and roadside infrastructure) to detect the environmental conditions referenced above.

To learn a driver's preferred following gaps across these different human and environmental contexts, vehicle 200 can correlate human context data and environmental data collected within a respective time interval with: (1) contemporaneous data (i.e., data from the same time interval) indicating speeds of vehicle 200 and following gaps following other vehicles during operation of an ACC system in vehicle 200; and (2) contemporaneous driving inputs provided by the driver during operation of the ACC system (e.g., the driver setting/pre-defining following gaps at different speeds, accelerator and brake pedal depression patterns, etc.). Such correlating may generate time-series data that vehicle 200 uses to dynamically compute (i.e., in real-time, or approximately real-time) the personalized ACC parameter.

In certain implementations, vehicle 200 may store the above-described data in an on-board memory resource (e.g., a data lake). Prior to computing the personalized ACC parameter, vehicle 200 may also process the data so it is transformed into a more suitable/machine readable format. For example, vehicle 200 may utilize one or more ML models to label or extract features from the data. Examples of such ML models may include: (i) one or more human context classifier models (e.g., a driver physiological state classifier, a driver psychological state classifier, etc.); (ii) one or more environmental context classifiers (e.g., a weather condition classifier, a time of day classifier, a traffic scenario classier, a road type classifier, etc.); (iii) one or more driving input classifiers (e.g., an acceleration and brake pattern classifier); (iv) an anomaly detection classifier; etc. As alluded to above, vehicle 200 can also correlate these different types of data in time to generate time series data. In such implementations, vehicle 200 may utilize the above-described data processing to compute the personalized ACC parameter more efficiently and accurately. In some implementations, vehicle 200 may utilize an ML model to compute the personalized ACC parameter based on the above-described data. In other implementations however, vehicle 200 may compute the personalized ACC parameter using other/non-ML-based techniques.

In various implementations, vehicle 200 may send the above-described data to an external computing resource (e.g., a cloud-based resource, an infrastructure-based resource, or a combination thereof). In accordance with various embodiments of the presently disclosed technology, the external computing resource may store the data in a data lake, along with similar data received from other vehicles. The external computing resource may perform the same/similar processing of the stored data as described in the previous paragraph. Accordingly, in certain implementations the external computing resource can use this processed data to train an ML model to compute personalized ACC parameters. In some of such implementations, a trained instance of this ML model may be implemented in vehicle 200 to compute the personalized ACC parameter of operation 502.

In some implementations, the personalized ACC parameter may comprise one or more safety-related boundaries. For example, the personalized ACC parameter may include an upper boundary that limits/prevents unsafe operation of vehicle 200 (e.g., from effectuating following gaps below a threshold level).

Referring now to operation 504, during at least at portion of ACC operation of vehicle 200, vehicle 200 can perform operation 504 to use the (computed) personalized ACC parameter to compute a target following gap for following a second vehicle.

In certain implementations, using the personalized ACC parameter to compute the target following gap may comprise using a feed forward function to compute the target following gap based on: (a) the personalized ACC parameter; (b) a current speed of vehicle 200; (c) current environmental conditions (e.g., as detected by sensors of vehicle 200); and (d) at least one of a current physiological state of the driver and a current psychological state of the driver (e.g., as detected by sensors of vehicle 200).

As depicted, vehicle 200 can then perform operation 506 to control speed of vehicle 200 in accordance with the (computed) target following gap. As alluded to above, this may involve vehicle 200 automatically controlling throttle or braking of vehicle 200.

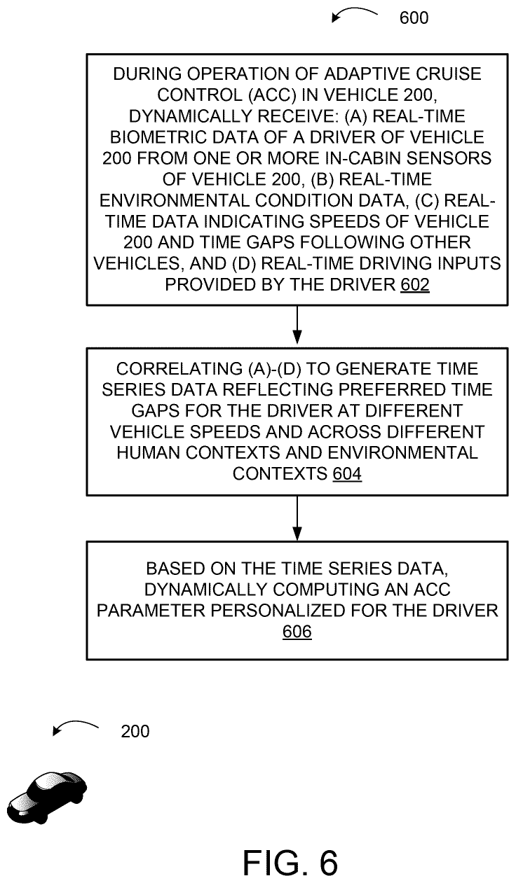

FIG. 6 depicts an example process 600 for computing a multi-dimensional personalized ACC parameter, in accordance with various embodiments of the presently disclosed technology. As depicted, vehicle 200 from FIG. 2 may be configured to perform process 600.

For example, during operation of an ACC system in vehicle 200, vehicle 200 may perform operation 602 to dynamically receive: (a) real-time biometric data of a driver of vehicle 200 from one or more in-cabin sensors of vehicle 200; (b) real-time environmental condition data (e.g., from one or more environmental and other sensors of vehicle 200); (c) real-time data indicating speeds of the vehicle and following gaps following other vehicles (e.g., from one or more environmental and other sensors of vehicle 200); and (d) real-time driving inputs provided by the driver.

Vehicle 200 may perform this operation in the same/similar manner as described in conjunction with operation 502 of process 500.

Vehicle 200 may perform operation 604 to correlate the above-described data (a)-(d) to generate time series data reflecting preferred following gaps for the driver at different vehicle speeds across different human contexts and environmental contexts. Vehicle 200 may perform this operation in the same/similar manner as described in conjunction with operation 502 of process 500.

Based on the time series data, vehicle 200 can perform operation 606 to dynamically compute an ACC parameter personalized for the driver (i.e., a personalized ACC parameter). As described above, the personalized ACC parameter may comprise a multi-dimensional vector reflecting the preferred following gaps for the driver at different vehicle speeds and across different human contexts and environmental contexts. Vehicle 200 may perform this operation in the same/similar manner as described in conjunction with operation 502 of process 500.

As described above, in certain implementations vehicle 200 may perform further operations to: (1) use the (computed) personalized ACC parameter to compute a target following gap for following a second vehicle; and (2) control speed of vehicle 200 in accordance with the (computed) target following gap. Vehicle 200 may perform these operations in the same/similar manner as described in conjunction with operations 504 and 506 of process 500 respectively.

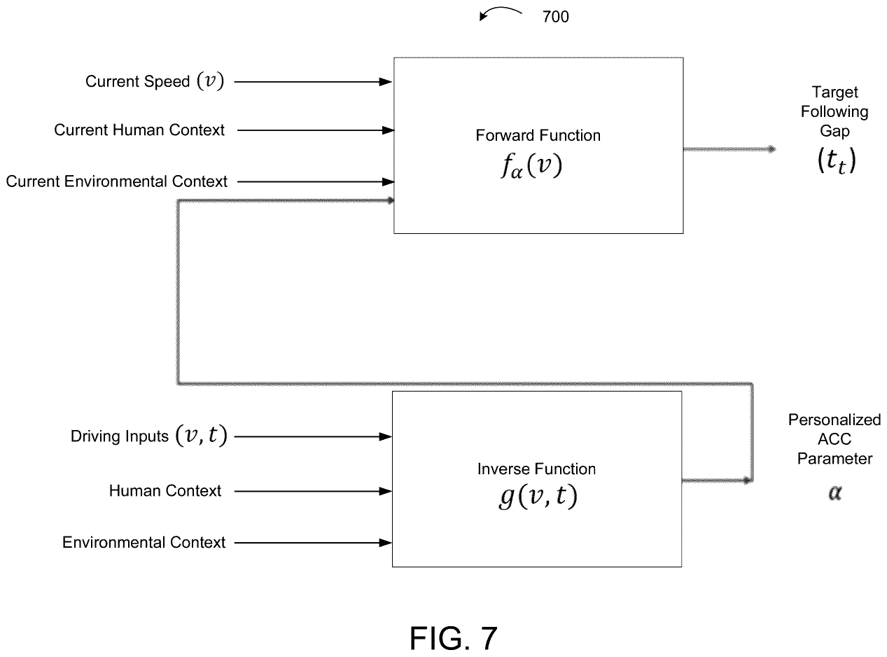

FIG. 7 depicts an example process 700 for computing a multi-dimensional personalized ACC parameter and using the multi-dimensional personalized ACC parameter to compute a target following gap, in accordance with various embodiments of the presently disclosed technology.

As depicted, process 700 may involve dynamically providing driving inputs, human context data, and environmental context data to an inverse function g.

As depicted, in certain implementations the driving inputs may comprise following gaps (t) a driver is setting/pre-defining at different vehicle speeds (v). In some of these implementations, the driving inputs may also comprise brake and accelerator pedal patterns of the driver at these following gaps and vehicle speeds.

As depicted, the inverse function g may dynamically compute a personalized ACC parameter α based on the above-described inputs. The personalized ACC parameter α may comprise a multi-dimensional vector reflecting the driver's desired following gaps at different vehicle speeds and across different human and environmental contexts.

As Described, by dynamically computing personalized ACC parameter α in response to new/real-time driving inputs, human context data, and environmental context data—process 700 can constantly refine the personalized ACC parameter α in response to this new/real-time data. In some implementations, inverse function g can weight more recent input data greater than relatively less recent data when computing the personalized ACC parameter α. In this way, process 700 can more effectively adjust to a driver's style/preferences as the driver's style/preferences evolve over time.

As depicted, process 700 may further involve providing the personalized ACC parameter α, a current vehicle speed, current human context data, and current environmental context data to a forward function f. Based on these inputs, the forward function f can compute a target following gap (tₜ). Accordingly, a vehicle implementing process 700 (e.g., vehicle 200) can control speed of the vehicle in accordance with the (computed) target following gap (tₜ).

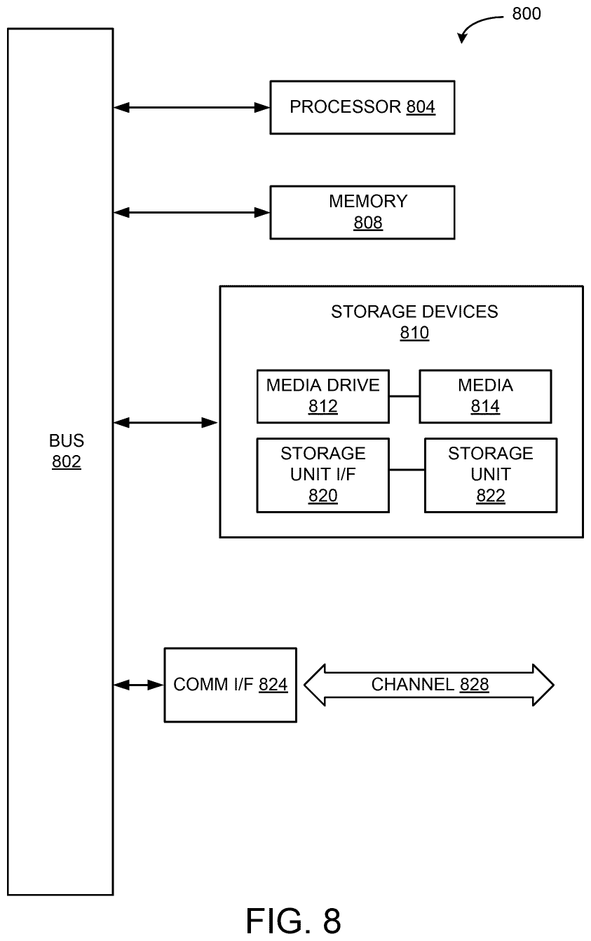

As used herein, the terms circuit, system, and component might describe a given unit of functionality that can be performed in accordance with one or more applications of the present application. As used herein, a component might be implemented utilizing any form of hardware, software, or a combination thereof. For example, one or more processors, controllers, ASICs, PLAs, PALs, CPLDs, FPGAs, logical components, software routines or other mechanisms might be implemented to make up a component. Various components described herein may be implemented as discrete components or described functions and features can be shared in part or in total among one or more components. In other words, as would be apparent to one of ordinary skill in the art after reading this description, the various features and functionality described herein may be implemented in any given application. They can be implemented in one or more separate or shared components in various combinations and permutations. Although various features or functional elements may be individually described or claimed as separate components, it should be understood that these features/functionality can be shared among one or more common software and hardware elements. Such a description shall not require or imply that separate hardware or software components are used to implement such features or functionality.

Where components are implemented in whole or in part using software (such as user device applications described herein), these software elements can be implemented to operate with a computing or processing component capable of carrying out the functionality described with respect thereto. One such example computing component is shown in FIG. 8. Various applications are described in terms of this example-computing component 800. After reading this description, it will become apparent to a person skilled in the relevant art how to implement the application using other computing components or architectures.

Referring now to FIG. 8, computing component 800 may represent, for example, computing or processing capabilities found within a vehicle (e.g., vehicle 150, vehicle 200), user device, self-adjusting display, desktop, laptop, notebook, and tablet computers. They may be found in hand-held computing devices (tablets, PDA's, smart phones, cell phones, palmtops, etc.). They may be found in workstations or other devices with displays, servers, or any other type of special-purpose or general-purpose computing devices as may be desirable or appropriate for a given application or environment. Computing component 800 might also represent computing capabilities embedded within or otherwise available to a given device. For example, a computing component might be found in other electronic devices such as, for example, portable computing devices, and other electronic devices that might include some form of processing capability. In another example, a computing component might be found in components making up vehicle 150, vehicle 200, personalized ACC circuit 310, decision and control circuit 303, computing system 100, computing system 210, ECU 225, etc.

Computing component 800 might include, for example, one or more processors, controllers, control components, or other processing devices. This can include a processor, and any one or more of the components making up vehicle 150 of FIG. 1, vehicle 200 of FIG. 2, computing system 210 of FIG. 2, and personalized ACC system 300 of FIG. 3. Processor 804 might be implemented using a general-purpose or special-purpose processing engine such as, for example, a microprocessor, controller, or other control logic. The processor 804 might be specifically configured to execute one or more instructions for execution of logic of one or more circuits described herein, such as personalized ACC circuit 310, decision and control circuit 303, and logic for control systems 240. Processor 804 may be configured to execute one or more instructions for performing one or more methods, such as the processes described in FIGS. 5-6.

Processor 804 may be connected to a bus 802. However, any communication medium can be used to facilitate interaction with other components of computing component 800 or to communicate externally. In applications, processor 804 may fetch, decode, and execute one or more instructions to control processes and operations for enabling vehicle servicing as described herein. For example, instructions can correspond to steps for performing one or more steps of the process described in FIGS. 5-6.

Computing component 800 might also include one or more memory components, simply referred to herein as main memory 808. For example, random access memory (RAM) or other dynamic memory, might be used for storing information and instructions to be fetched, decoded, and executed by processor 804. Such instructions may include one or more instructions for execution of one or more logical circuits described herein. Instructions can include instructions 208 of FIG. 2, and instructions 309 of FIG. 3 as described herein, for example. Main memory 808 might also be used for storing temporary variables or other intermediate information during execution of instructions to be fetched, decoded, and executed by processor 804. Computing component 800 might likewise include a read only memory (“ROM”) or other static storage device coupled to bus 802 for storing static information and instructions for processor 804.

The computing component 800 might also include one or more various forms of information storage mechanism 810, which might include, for example, a media drive 812 and a storage unit interface 820. The media drive 812 might include a drive or other mechanism to support fixed or removable storage media 814. For example, a hard disk drive, a solid-state drive, a magnetic tape drive, an optical drive, a compact disc (CD) or digital video disc (DVD) drive (R or RW), or other removable or fixed media drive might be provided. Storage media 814 might include, for example, a hard disk, an integrated circuit assembly, magnetic tape, cartridge, optical disk, a CD or DVD. Storage media 814 may be any other fixed or removable medium that is read by, written to or accessed by media drive 812. As these examples illustrate, the storage media 814 can include a computer usable storage medium having stored therein computer software or data.

In alternative applications, information storage mechanism 810 might include other similar instrumentalities for allowing computer programs or other instructions or data to be loaded into computing component 800. Such instrumentalities might include, for example, a fixed or removable storage unit 822 and an interface 820. Examples of such storage unit 822 and interface 820 can include a program cartridge and cartridge interface, a removable memory (for example, a flash memory or other removable memory component) and memory slot. Other examples may include a PCMCIA slot and card, and other fixed or removable storage units 822 and interfaces 820 that allow software and data to be transferred from storage unit 822 to computing component 800.

Computing component 800 might also include a communications interface 824. Communications interface 824 might be used to allow software and data to be transferred between computing component 800 and external devices. Examples of communications interface 824 might include a modem or softmodem, a network interface (such as Ethernet, network interface card, IEEE 802.XX or other interface). Other examples include a communication port (such as for example, a USB port, IR port, RS232 port Bluetooth® interface, or other port), or other communications interface. Software/data transferred via communications interface 824 may be carried on signals, which can be electronic, electromagnetic (which includes optical) or other signals capable of being exchanged by a given communications interface 824. These signals might be provided to communications interface 824 via a channel 828. Channel 828 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media. Such media may be, e.g., memory 808, storage unit 822, media 814, and channel 828. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 800 to perform features or functions of the present application as discussed herein.

As described herein, vehicles can be flying, partially submersible, submersible, boats, roadway, off-road, passenger, truck, trolley, train, drones, motorcycle, bicycle, or other vehicles. As used herein, vehicles can be any form of powered or unpowered transport. Obstructions can include one or more potholes, cracks, tire markings, faded road markings, debris, objects, occlusion, road reflection, floodings, icy surfaces, oil leaks, uneven pavement, erosions, raveling and other potentially hazardous conditions on the road. Although roads are references herein, it is understood that the present disclosure is not limited to roads or to 1d or 2d traffic patterns.

The term “operably connected,” “coupled”, or “coupled to”, as used throughout this description, can include direct or indirect connections, including connections without direct physical contact, electrical connections, optical connections, and so on.

The terms “a” and “an,” as used herein, are defined as one or more than one. The term “plurality,” as used herein, is defined as two or more than two. The term “another,” as used herein, is defined as at least a second or more. The terms “including” and “having,” as used herein, are defined as comprising (i.e., open language). The phrase “at least one of . . . and . . . ” as used herein refers to and encompasses any and all possible combinations of one or more of the associated listed items. As an example, the phrase “at least one of A, B, or C” includes A only, B only, C only, or any combination thereof (e.g., AB, AC, BC or ABC).

Aspects herein can be embodied in other forms without departing from the spirit or essential attributes thereof. Accordingly, reference should be made to the following claims, rather than to the foregoing specification, as indicating the scope hereof. While various applications of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent module names other than those depicted herein can be applied to the various partitions. Additionally, with regard to flow diagrams, operational descriptions and method claims, the order in which the steps are presented herein shall not mandate that various applications be implemented to perform the recited functionality in the same order, and with each of the steps shown, unless the context dictates otherwise.

Although the disclosed technology is described above in terms of various exemplary applications and implementations, it should be understood that the various features, aspects and functionality described in one or more of the individual applications are not limited in their applicability to the particular application with which they are described, but instead can be applied, alone or in various combinations, to one or more of the other applications of the disclosed technology, whether or not such applications are described and whether or not such features are presented as being a part of a described application. Thus, the breadth and scope of the technology disclosed herein should not be limited by any of the above-described exemplary applications.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various applications set forth herein are described in terms of exemplary block diagrams, flow charts and other illustrations. As will become apparent to one of ordinary skill in the art after reading this document, the illustrated applications and their various alternatives can be implemented without confinement to the illustrated examples. For example, block diagrams and their accompanying description should not be construed as mandating a particular architecture or configuration.

What is claimed is:

1. A method comprising:
computing an Adaptive Cruise Control (ACC) parameter personalized for a driver of a vehicle, wherein the personalized ACC parameter comprises a multi-dimensional vector reflecting preferred following gaps for the driver across different human contexts and environmental contexts;
during at least a portion of ACC operation of the vehicle, using the personalized ACC parameter to compute a target following gap for following a second vehicle; and
controlling speed of the vehicle in accordance with the target following gap.

2. The method of claim 1, wherein the multi-dimensional vector comprises:
one or more dimensions reflecting the human contexts; and
one or more dimensions reflecting the environmental contexts.

3. The method of claim 2, wherein the multi-dimensional vector has different values at different vehicle speeds.

4. The method of claim 2, wherein the one or more dimensions reflecting the human contexts comprise one or more dimensions reflecting at least one of:
physiological states of the driver; and
psychological states of the driver.

5. The method of claim 2, wherein the one or more dimensions reflecting the environmental contexts comprise one or more dimensions reflecting at least one of:
times of day;
weather conditions;
traffic scenarios; and
road types.

6. The method of claim 3, wherein computing the personalized ACC parameter for the driver comprises computing the personalized ACC parameter dynamically based on:
real-time speeds of the vehicle and corresponding following gaps behind other vehicles when the ACC is enabled;
real-time driving inputs provided by the driver;
real-time environmental conditions; and
at least one of real-time physiological states of the driver and real-time psychological states of the driver.

7. The method of claim 6, wherein using the personalized ACC parameter to compute the target following gap for following the second vehicle comprises using a feed forward function to compute the target following gap based on:
the personalized ACC parameter;
a current speed of the vehicle;
current environmental conditions; and
at least one of a current physiological state of the driver and a current psychological state of the driver.

8. The method of claim 1, wherein the personalized ACC parameter comprises one or more safety-related boundaries.

9. A vehicle comprising:
one or more processors; and
memory storing instructions, which when executed by the one or more processors, cause the vehicle to perform operations, the operations comprising:
computing an Adaptive Cruise Control (ACC) parameter personalized for a driver of the vehicle, wherein the personalized ACC parameter comprises a multi-dimensional vector reflecting preferred following gaps for the driver across different human contexts and environmental contexts;
during at least a portion of ACC operation of the vehicle, using the personalized ACC parameter to compute a target following gap for following a second vehicle; and
controlling speed of the vehicle in accordance with the target following gap.

10. The vehicle of claim 9, wherein the multi-dimensional vector comprises:
one or more dimensions reflecting the human contexts; and
one or more dimensions reflecting the environmental contexts.

11. The vehicle of claim 10, wherein the one or more dimensions reflecting the human contexts comprise one or more dimensions reflecting at least one of:
physiological states of the driver; and
psychological states of the driver.

12. The vehicle of claim 11, wherein the operations further comprise:
receiving, from a device of the driver, biometric data of the driver; and
based on the received biometric data, determining at least one of the physiological states of the driver and the psychological states of the driver.

13. The vehicle of claim 11, further comprising one or more in-cabin sensors, wherein the operations further comprise:
receiving, from the one or more in-cabin sensors, biometric data of the driver; and
based on the received biometric data, determining at least one of the physiological states of the driver and the psychological states of the driver.

14. The vehicle of claim 13, wherein the one or more in-cabin sensors comprise at least one of:
a camera positioned to capture facial expressions of the driver;
an electromyography sensor; and
a heart rate sensor.

15. The vehicle of claim 10, wherein the one or more dimensions reflecting the environmental contexts comprise one or more dimensions reflecting at least one of:
times of day;
weather conditions;
traffic scenarios; and
road types.

16. The vehicle of claim 10, wherein the multi-dimensional vector comprises different values at different vehicle speeds and computing the personalized ACC parameter for the driver comprises computing the personalized ACC parameter dynamically based on:
real-time speeds of the vehicle and corresponding following gaps behind other vehicles when the ACC is enabled;
real-time driving inputs provided by the driver;
real-time environmental conditions; and
at least one of real-time physiological states of the driver and real-time psychological states of the driver.

17. The vehicle of claim 16, wherein using the personalized ACC parameter to compute the target following gap for following the second vehicle comprises using a feed forward function to compute the target following gap based on:
the personalized ACC parameter;
a current speed of the vehicle;
current environmental conditions; and
at least one of a current physiological state of the driver and a current psychological state of the driver.

18. A vehicle comprising:
one or more in-cabin sensors;
one or more processors; and
memory storing instructions, which when executed by the one or more processors, cause the vehicle to perform operations, the operations comprising:
during operation of adaptive cruise control (ACC) in the vehicle, dynamically receiving:
(a) real-time biometric data of a driver of the vehicle from the one or more in-cabin sensors,
(b) real-time environmental condition data,
(c) real-time data indicating speeds of the vehicle and following gaps following other vehicles, and
(d) real-time driving inputs provided by the driver;
correlating (a)-(d) to generate time series data reflecting preferred following gaps for the driver at different vehicle speeds across different human contexts and environmental contexts; and
based on the time series data, dynamically computing an ACC parameter personalized for the driver, wherein the personalized ACC parameter comprises a multi-dimensional vector reflecting the preferred following gaps for the driver at different vehicle speeds across different human contexts and environmental contexts.

19. The vehicle of claim 18, wherein the operations further comprise dynamically determining, based on the received biometric data, at least one of:
real-time physiological states of the driver; and
real-time psychological states of the driver.

20. The vehicle of claim 18, wherein the one or more in-cabin sensors comprise at least one of:
a camera positioned to capture facial expressions of the driver;
an electromyography sensor; and
a heart rate sensor.
